(19) United States
(12) Patent Application Publication
(10) Pub. No.: US 2008/0262464 A1
(43) Pub. Date: Oct. 23, 2008

(54) Tampon and method and apparatus for making same

(75) Inventors: Margaret Henderson Hasse, Wyoming, OH (US); Michael Henry Morris, West Chester, OH (US); Lloyd Keith Stephenson, Burlington, KY (US); Daniel Raymond Wiegele, Cincinnati, OH (US)

Correspondence Address: THE PROCTER & GAMBLE COMPANY;Global Legal Department - IP, Sycamore Building - 4th Floor, 299 East Sixth Street, CINCINNATI, OH, 45202, US

(73) Assignee: The Procter & Gamble Company

(21) Appl. No.: 11/787,819

(22) Filed: Apr. 18, 2007

(51) Int. Cl. A61F 13/20; B29C 65/00

(52) U.S. Cl. 604/385.18; 156/60

(57) ABSTRACT

A tampon compressed with a compression having a major component in a widthwise direction may have a plurality of creases extending along its length from its surface and penetrating no deeper than about 20% of the width of the tampon. A method for making a tampon may comprise compressing an uncompressed pledget in a compression machine and feeding the compressed pledget with a compression member into a tampon mold having a mold cavity for receiving the compressed pledget, the compressed pledget thickness being no more that about 20% different than the uncompressed pledget thickness and the tampon mold having a mold cavity thickness no more than about 10% different than the thickness of the compressed pledget. An apparatus for making a tampon is also disclosed.

### FIELD OF THE INVENTION

The invention relates to improved tampons and to apparatuses and methods of making such tampons.

### BACKGROUND OF THE INVENTION

Tampons are generally compressed absorbent structures typically shaped and sized to fit into a body cavity, such as, for example a human vagina. Often, due to the processes used for making tampons, the outer surface of the tampon may be relatively rough, uneven, crenulated, and unattractive. Such characteristics in the surface topography of a tampon may provide the user with a negative impression of the product, especially when seen prior to use. For example, certain tampons may be provided to the used for digital insertion and others may be provided in applicators that at least partially display the outer surface of the product. Further, the methods used to provide current tampons may inhibit certain performance benefits, such as, for example, the ability for the tampon to expand during use.

Accordingly, it would be desirable to provide tampons that have a relatively smoother and/or less crenulated outer surface. It may also be advantageous to provide a tampon that has a more attractive appearance prior to use. Further, it may be desirable to provide a tampon that has improved expansion characteristics during use. Further still, it would be desirable to provide an apparatus and/or method of making a tampon that provides a tampon with a relatively smoother tampon outer surface, improves the appearance of the outer surface of the tampon prior to use and/or provides the tampon with improved expansion characteristics during use.

### SUMMARY OF THE INVENTION

The present invention addresses one or more of the foregoing technical problems and provides a tampon which may comprise a compressed pledget of absorbent material. The tampon may have a surface, a length extending from a withdrawal end to an insertion end distal from the withdrawal end, a width extending substantially perpendicularly with respect to the length, a thickness extending substantially perpendicularly with respect to the length and the width, and a plurality of creases extending along the length from the surface of the tampon and penetrating no deeper than about 20% of the tampon width. The compressed pledget may have been compressed with a major component in a widthwise direction with respect to the compressed or finished tampon and no more than a minor component in lengthwise and thickness directions with respect to the compressed or finished tampon.

According to another aspect of this invention, an apparatus for making a tampon is provided and may comprise a compression machine for receiving and compressing an uncompressed pledget of absorbent material, a tampon mold having a mold cavity for receiving a compressed pledget from the compression machine, and a compression member for pushing the compressed pledget into the tampon mold cavity. The uncompressed pledget may have an uncompressed pledget length extending from an insertion end to a withdrawal end, an uncompressed pledget width substantially perpendicular to the uncompressed pledget length, and an uncompressed pledget thickness extending substantially perpendicularly to both the uncompressed pledget length and the uncompressed pledget width. The compression machine may compress the uncompressed pledget with a compression having a major component in a widthwise direction with respect to the uncompressed pledget and no more than a minor component in lengthwise and thickness directions so as to form a compressed pledget having a compressed pledget length extending from the insertion end to the withdrawal end, a compressed pledget width substantially perpendicular to the compressed pledget length, and a compressed pledget thickness extending substantially perpendicularly to both the compressed pledget length and the compressed pledget width. Furthermore, the mold cavity of the tampon mold may have a mold cavity length extending from an opening to a distal end, a mold cavity width extending substantially perpendicularly to the mold cavity length, and a mold cavity thickness extending perpendicularly to both the mold cavity length and mold cavity width. The mold cavity thickness may be no more than about 10% different than the compressed pledget thickness.

According to yet another aspect of this invention, a method is provided for making a tampon which may comprise providing an uncompressed pledget of absorbent material, compressing the uncompressed pledget in a compression machine, feeding the compressed pledget with a compression member to a tampon mold having a mold cavity for receiving the compressed pledget from the compression machine. The pledget may have an uncompressed pledget length extending from an insertion end to a withdrawal end, an uncompressed pledget width substantially perpendicular to the uncompressed pledget length, and an uncompressed pledget thickness extending substantially perpendicularly to both the uncompressed pledget length and the uncompressed pledget width. The uncompressed pledget may be compressed in a compression machine with a compression having a major component in a widthwise direction with respect to the pledget and no more than a minor component in lengthwise and thickness directions so as to form a compressed pledget having a compressed pledget length extending from the insertion end to the withdrawal end, a compressed pledget width substantially perpendicular to the compressed pledget length, and a compressed pledget thickness extending substantially perpendicularly to both the compressed pledget length and the compressed pledget width, the compressed pledget thickness being no more than about 20% different than the uncompressed pledget thickness. The mold cavity may have a mold cavity length extending from an opening to a distal end, a mold cavity width extending substantially perpendicularly to the mold cavity length, and a mold cavity thickness extending perpendicularly to both the mold cavity length and the mold cavity width, the mold thickness being no more than about 10% different than the compressed pledget thickness.

Other features and advantages of the invention may be apparent from reading the following detailed description, drawings, and claims.

### BRIEF DESCRIPTION OF THE DRAWINGS

FIG. 1 is a perspective view of an uncompressed pledget of absorbent material for use in making a tampon in accordance with an embodiment of the present invention.

FIG. 2 is a plan view of a tampon forming apparatus, in accordance with an embodiment of the present invention, with the tampon compression machine in an open position.

FIG. 3 is a perspective view of the tampon compression machine of FIG. 2 in an open position.

FIG. 4 is a perspective view of the tampon compression machine of FIG. 2 in a compression position.

FIG. 5 is a partial perspective view of the compression member which forms part of the tampon forming apparatus in FIG. 2.

FIG. 6 is a perspective view of a split cavity tampon mold which is part of the tampon forming apparatus illustrated in FIG. 2.

FIG. 7 is partial perspective view of the tampon forming apparatus in FIG. 2 with the tampon compression machine in open position and an uncompressed pledget in the tampon compression machine cavity.

FIG. 8 is a partial perspective view of the tampon forming apparatus of FIG. 2 with the tampon compression machine in a final compression position and a compressed pledget in the compression machine cavity.

FIG. 9 is a partial perspective view of the tampon forming apparatus of FIG. 2 with a compressed pledget in the tampon split mold cavity.

FIG. 10 is a perspective view a tampon made in accordance with an embodiment of this invention.

FIG. 11 is a perspective view of a tampon made in accordance with the prior art.

FIG. 12 is a frontal elevation view of a syngyna test apparatus used to conduct the Expansion Under Pressure Test described herein below.

FIG. 13 is a side elevation view of the test apparatus shown in FIG. 12.

### DETAILED DESCRIPTION OF THE INVENTION

As summarized above, the present invention may encompass a tampon and an apparatus and method for making such a tampon. As will be explained in more detail below, tampons in accordance with embodiments of the invention may be made by compressing an uncompressed pledget of absorbent material with a tampon forming apparatus having relatively close tolerances so that creases in the surface of the tampon are many and/or relatively shallow. Allowing relatively little movement of a pledget in the direction of pledget thickness during compression in the direction of the pledget width may focus energy in the direction of the pledget width. As a result, tampons made in accordance with certain embodiments of this invention may have an improved surface appearance and/or improved re-expansion abilities.

Section A below describes terms for assisting the reader in understanding features of the invention, but not introducing limitations in the terms inconsistent with the context with which they are used in the specification. Section B is a detailed description of the drawings illustrating an apparatus in accordance with embodiments of this invention. Section C describes methods of manufacturing tampons in accordance with embodiments of this invention and Section D describes tampons made in accordance with this invention.

A. Terms

As used herein, “compression” refers to the process of pressing, squeezing, compacting or otherwise manipulating the size, shape, and/or volume of a material to obtain a tampon having a vaginally insertable shape. The term “compressed” refers to the state of a material or materials subsequent to compression. Conversely, the term “uncompressed” refers to the state of a material or materials prior to compression. The term “compressible” is the ability of a material to undergo compression.

As used herein, “mold” refers to a structure for shaping a pledget during compression and/or retaining the shape for a compressed pledget subsequent to compression during the stabilization process. Molds have an inner surface defining an inner cavity and an outer surface. The inner cavity is structured to define or mirror the shape of the compressed absorbent pledget. Thus, in some embodiments the pledget conforms to the shape of the inner cavity of the mold by a restraining force to result in a self-sustaining shape and is retained in the inner cavity during the stabilization process. In other embodiments, the mold retains the shape of the compressed pledget during the stabilization process. The inner cavity may be profiled to achieve any shape known in the art including, but not limited to, cylindrical, oval, rectangular, triangular, trapezoidal, semi-circular, hourglass, serpentine or other suitable shapes. The outer surface of the mold is the surface external to the inner surface and can be profiled or shaped in any manner, such as, rectangular, cylindrical or oblong. The mold may comprise one or more members. Suitable molds used in the present invention may include, but may not be limited to unitary molds, comprising one member, and split cavity molds. Examples of split cavity molds include those disclosed in U.S. patent application Ser. No. 10/150,050 entitled “Substantially Serpentine Shaped Tampon,” and U.S. patent application Ser. No. 10/150,055, entitled “Shaped Tampon,” both filed on Mar. 18, 2002.

As used herein the term “pledget” refers to a construction of absorbent material prior to the compression of such construction into a tampon.

As used herein, “self-sustaining” is a measure of the degree or sufficiency to which the tampon retains its compressed form after stabilization such that in the subsequent absence of external forces, the resulting tampon will tend to retain its vaginally insertable shape and size. It will be understood by one of skill in the art that this self-sustaining form need not, and may not persist during actual use of the tampon. That is, once the tampon is inserted into the vagina or other body cavity and begins to acquire fluid, the tampon will begin to expand and may lose its self-sustaining form.

The term “shaped tampons,” as used herein, refers to compressed pledgets having either a substantially serpentine shape, an “undercut” or “waist,” or a non-uniform cross-section traversing from the insertion end to the withdrawal end of the tampon. The phrase “substantially serpentine” refers to a non-linear dimension between any two points spaced at least about 5 mm apart. The term “undercut” refers to tampons having a protuberance or indentation that impedes the withdrawal from a unitary mold. For example, shaped tampons may be hourglass shaped having at least one perimeter in the center of the tampon or “waist” that is less than both an insertion end perimeter and a withdrawal end perimeter.

As used herein, the term “split cavity mold” is a mold comprised of two or more members that when brought together complete the inner cavity of the mold. Each member of the split cavity mold comprises at least a portion of the inner surface that when brought together or closed completes the mold structure. The split cavity mold is designed such that at least two or more of the mold members can be at least partially separated, if not fully separated, typically after the tampon has acquired a self-sustaining shape, to expand the cavity volume circumscribed by the inner surface(s) thus permitting the easier removal of the tampon from the mold. Partial separation can occur when only a portion of two mold members are separated while other portions of the two mold members remain in contact. Where each member's inner surface portion joins the inner surface portion of another member, those points of adjacency can define a straight line, a curve, or another seam of any convoluted intersection or seam of any regular or irregular form. The elements of the split cavity in some embodiments may be held in appropriate position relative to each other by linking elements of any form including bars, rods, linked cams, chains, cables, wires, wedges, screws, etc.

The term “stabilized,” as used herein, refers to a tampon in a self-sustaining state wherein it has overcome the natural tendency to re-expand to the original size, shape and volume of the absorbent material and overwrap, which comprise the pledget.

As used herein the term “tampon,” refers to any type of absorbent structure that is inserted into the vaginal canal or other body cavity for the absorption of fluid therefrom, to aid in wound healing, or for the delivery of active materials, such as medicaments, or moisture. The tampon may be compressed into a generally cylindrical configuration in the radial direction, axially along the longitudinal axis or in both the radial and axial directions. While the tampon may be compressed into a substantially cylindrical configuration, other shapes are possible. These may include shapes having a cross section that may be described as oval, rectangular, triangular, trapezoidal, semi-circular, hourglass, serpentine, or other suitable shapes. Tampons have an insertion end, withdrawal end, a length, a width, a longitudinal axis and a radial axis. The tampon's length can be measured from the insertion end to the withdrawal end along the longitudinal axis. A typical compressed tampon for human use is within a range from about 30 mm to about 60 mm in length. A tampon may be straight or non-linear in shape, such as curved along the longitudinal axis. A typical compressed tampon is within a range from about 8 mm to about 20 mm wide. The width of a tampon, unless otherwise stated in the specification, corresponds to the distance across the largest cross-section, along the length of the tampon and perpendicular to the longitudinal axis of the tampon.

The term “vaginal cavity,” “within the vagina,” and “vaginal interior,” as used herein, are intended to be synonymous and refer to the internal genitalia of the mammalian female in the pudendal region of the body. The term “vaginal cavity” as used herein is intended to refer to the space located between the introitus of the vagina (sometimes referred to as the sphincter of the vagina or hymeneal ring,) and the cervix. The terms “vaginal cavity,” “within the vagina” and “vaginal interior,” do not include the interlabial space, the floor of vestibule or the externally visible genitalia.

As used herein, “cm” is centimeter, “g” is grams, “g/cc” is grams per cubic centimeter, “g/m²” is grams per meter squared, “L” is liters, “L/s” is liters per second, “mL” is milliliters”, “mm” is millimeters, “min” is minutes, “psi” is pounds per square inch, “rpm” is rate per minute, and “s” is seconds.

The term “crease” as used herein, is the configuration of the compressed pledget that may be incidental or deliberate to compaction of the pledget. The creased configuration may be characterized by at least one bend at least in a portion of the pledget such that portion of the pledget may be positioned with a different plane than before with the observation that the surface regions near the bend may be in a different distal and angular relationship to each other after the folding has taken place. The term “crease” encompasses folds and wrinkles. In the case of the lateral compaction of a generally flat pledget, there may exist one or more creases in the form of bends or folds of generally 180 degrees such that the surface regions on either side of the bend may be juxtaposed or even in co-facial contact with each other.

As used herein, the “tampon compression machine” is a machine assembly that includes parts that may compress a pledget. Typically a pledget compressed in the tampon compression machine is then transferred to a mold for final shaping into a self-sustaining form of a vaginally insertable shape where, the mold may further compress parts of the pledget beyond that which the tampon compression machine accomplished prior.

As used herein, the “compression member” is any member that can be used to compress a pledget. It can also function to transfer a compressed pledget.

As used herein, actuating is any force delivered by an electric motor, mechanical transmission, pneumatically, linear drive, manual, and/or hydraulic.

As used herein, a high aspect ratio shape is any shape in which the length is greater than the diameter or width of the shape. The shape may not necessarily contain any defined circles, arcs, or cross-sectional portions.

B. Tampon Manufacturing Apparatus

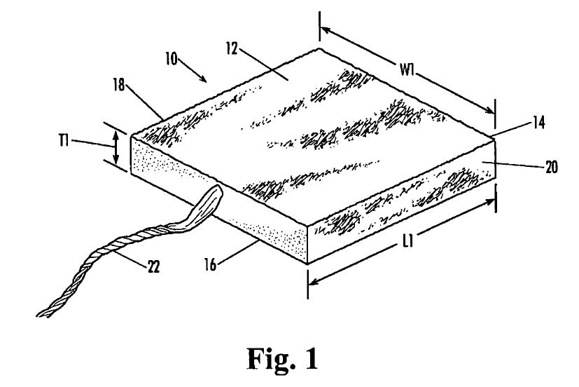

Turning to FIG. 1, an uncompressed pledget 10 of absorbent material 12 is illustrated. The uncompressed pledget 10 may be compressed to form a tampon in accordance with an embodiment of this invention. The uncompressed pledget 10 extends from an insertion end 14 to a withdrawal end 16 with opposing sides 18 and 20 extending from the insertion end 14 to the withdrawal end 16. A withdrawal cord or drawstring 22 may be connected to and extend from the withdrawal end 16 of the uncompressed pledget 10.

Although the uncompressed pledget 10 is illustrated as having a generally square or rectangular shape, the uncompressed pledget 10 can have a variety of shapes including, but not limited to, oval, round, chevron, square, rectangular, and the like. The uncompressed pledget 10 may have a length L1 extending from the insertion end 14 to the withdrawal end 16 of the uncompressed pledget 10, a width W1 extending from the one side 18 of the uncompressed pledget 10 to the other side 20 and perpendicularly to the length L1, and a thickness T1 extending perpendicularly to both the length L1 and width W1 of the uncompressed pledget 10.

The absorbent material 12 of the uncompressed pledget 10 may be constructed from a wide variety of liquid absorbing materials commonly used in absorbent articles. Such materials include but are not limited to rayon (such as GALAXY rayon, SARILLE L rayon both available from Accordis Kelheim GmbH of Kelheim, Germany), cotton, folded tissues, woven materials, nonwoven webs, synthetic and/or natural fibers or sheathing, comminuted wood pulp which is generally referred to as airfelt, or combinations of these materials. Other materials that may be incorporated into the pledget 10 include peat moss, absorbent foams (such as those disclosed in U.S. Pat. No. 3,994,298 issued to Desmarais on Nov. 30, 1976 and U.S. Pat. No. 5,795,921 issued to Dyer, et al.), capillary channel fibers (such as those disclosed in U.S. Pat. No. 5,356,405 issued to Thompson, et al. issued on Oct. 18, 1994), high capacity fibers (such as those disclosed U.S. Pat. No. 4,044,766 issued to Kaczmarck, et al. on Aug. 30, 1994), and super absorbent polymers or absorbent gelling materials (such as those disclosed in U.S. Pat. No. 5,830,543 issued to Miyake, et al. on Nov. 3, 1998). A more detailed description of liquid absorbing materials can be found in U.S. Pat. No. 6,740,070 to Raymond Agyapong.

The uncompressed pledget 10 may optionally include an overwrap comprising materials such as rayon, cotton, bicomponent fibers, polyethylene, polypropylene, other suitable natural or synthetic fibers known in the art, and mixtures thereof. In some embodiments, the uncompressed pledget 10 has a nonwoven overwrap comprised of bicomponent fibers that have a polypropylene core surrounded by polyethylene manufactured by Vliesstoffwerke Christian Heinrich Sandler GmbH and Company KG (Schwarzenbach/Salle Germany) under the trade name SAS B31812000. In other embodiments, the tampon may comprise a nonwoven overwrap of a hydro entangled blend of 50% rayon, 50% polyester available as BBA 140027 produced by BBA Corporation of South Carolina, US. In certain embodiments, the overwrap may be treated to hydrophilic, hydrophobic, wicking or nonwicking.

The uncompressed pledget 10 may optionally include a secondary absorbent member, an additional overwrap, a skirt portion and/or an applicator. The withdrawal cord 22 attached to the uncompressed pledget 10 may be made of any suitable material in the prior art such as cotton and rayon. U.S. Pat. No. 6,258,075 issued to Taylor et al. describes a variety of secondary absorbent members for use in pledgets. An example of a skirt portion is disclosed in U.S. Pat. No. 6,840,927 to Margaret Hasse.

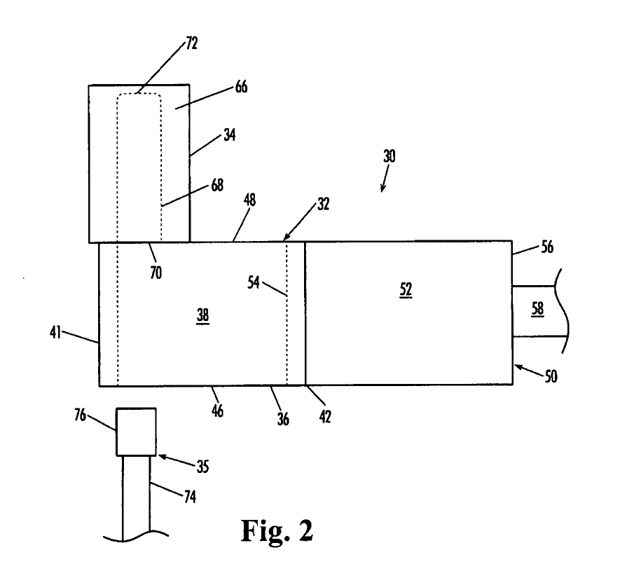

A tampon forming apparatus 30 for making tampons in accordance with an embodiment of this invention is illustrated in FIGS. 2-9. The tampon forming apparatus 30 may generally comprise a tampon compression machine 32 for initially compressing the uncompressed pledget 10 of absorbent material to form a compressed pledget 33, a split cavity mold 34 for receiving the compressed pledget 33 and setting the compressed pledget 33 in a self-sustaining shape, and a compression member 35 for pushing the compressed pledget 33 into the split cavity mold 34.

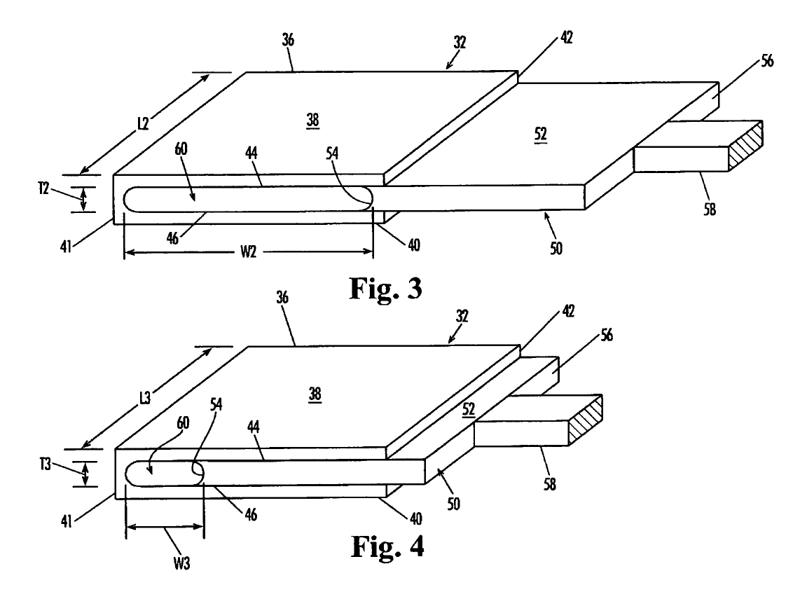

The tampon compression machine 32 may comprise a u-shaped anvil 36, as shown in FIGS. 3 and 4. The tampon compression machine 32 may comprise a top plate 38 and a juxtaposed bottom plate 40 extending from an end wall 41 connecting the top and bottom plates 38 and 40 to an open end 42 thereby forming a channel 44 between the top and bottom plates 38 and 40. The channel 44 may extend from an inlet end 46 to a discharge end 48 of the anvil 36. The tampon compression machine 32 may also comprise a die 50 comprising a solid plate 52 extending from a leading end 54 to a trailing end 56 and an actuating rod 58 connected to the trailing end 54 for reciprocating the die 50 within the channel 44 of the anvil 36. The leading end 54 of the die 50 and the top and bottom plates 38 and 40 and end wall 41 of the anvil 36 may form a compression machine cavity 60 within the channel 44 of the anvil 36 for receiving the uncompressed pledget 10. The die 50 may compress the uncompressed pledget 10 in the compression machine cavity to form the compressed pledget 33.

In certain embodiments, opposing plates 38 and 40 with end walls on opposing ends may move relative to each other and thereby compress the uncompressed pledget 10. Other configurations for the compression machine 32 for carrying out the functions described herein will be apparent to those skilled in the art from reading the details of this specification.

The compression machine cavity 60 of the tampon compression machine 32 may have an oval cross sectional shape as illustrated in FIGS. 3 and 4, but it should be understood that the compression machine cavity 60 may have other shapes as well including, but not limited to, round, square, and rectangular cross-sectional shapes.

When in an open configuration as illustrated in FIG. 3, the compression machine cavity 60 may have a length L2 extending from the inlet end 46 of the anvil 36 of the discharge end 48, a width W2 extending from the interior of the anvil end wall 41 to the leading end 54 of the die 50 and perpendicular to the length L2, and a thickness T2 extending from the interior of the top plate 38 of the anvil 36 to the bottom plate 40 perpendicular both to the length L2 and width W2 of the compression machine cavity 60. In some embodiments, the width W2 of the compression machine cavity 60 when the compression machine cavity is in an open configuration may be close to or greater than the width W2 of the uncompressed pledget 10. In certain embodiments, the length L2 of the compression machine cavity 60 may also be close to or greater than the length L1 of the uncompressed pledget 10 and the thickness T2 of the compression machine cavity 60 may be no more than about 20% different than the uncompressed pledget thickness T1, or no more than about 10% different than the uncompressed pledget thickness T1, or no more than about 5% different than the uncompressed pledget thickness T1. In certain embodiments when the thickness T2 of the compression machine cavity 60 is close to the same thickness T1 of the uncompressed pledget 10, the creases created in the surface of the compressed pledget 33 and the resulting tampon may be relatively shallow.

When in a compression configuration as illustrated in FIG. 4, the compression machine cavity 60 may have a length L3 which is the same as the length L2 in the open configuration and a thickness T3 which is same thickness as T2 in the open configuration, but may have a width W3 which may be substantially less than the width W2 of the compression machine cavity 60 in the open configuration and may be substantially less than the width W1 of the uncompressed pledget 10. In certain embodiments when the uncompressed pledget 10 is compressed in the tampon compression machine 32, the compressed pledget 33 may adopt the cross-sectional shape and width and thickness of the compression machine cavity 60 in the compressed configuration. Thus, the compressed pledget 33 may have a width of W3 and a thickness of T3. The manner of actuation of the die 50 within the anvil channel 44 to compress the pledget 10 may be by any suitable means to drive the actuating rod 58.

The degree of compression of the uncompressed pledget 10 in the compression machine cavity 60 in the widthwise direction may be a major component of the compression. In accordance with certain embodiments of this invention, the major compression of the uncompressed pledget in the compression machine cavity 60 in the widthwise direction is within a range from about 65% to about 90% of the original width of the uncompressed pledget 10. The degree of compression of the uncompressed pledget 10 in the thickness and lengthwise directions may be a minor component of the compression and, in accordance with certain embodiments of this invention, the minor compression of the uncompressed pledget 10 in the compression machine cavity 60 in the thickness and lengthwise directions may be no more than about 40% of the original width of the uncompressed pledget 10. In accordance with certain embodiments of this invention, the major compression of the uncompressed pledget 10 in the compression machine cavity 60 in the widthwise direction may be from about 75% to about 85% of the original width of the uncompressed pledget 10 and the minor compression of the uncompressed pledget 10 in the compression machine cavity 60 in the thickness and lengthwise directions may be no more than about 30% of the original width of the uncompressed pledget 10. It should be understood that it is contemplated in certain embodiments of the invention that there may be no compression of the uncompressed pledget 10 in the lengthwise and/or thickness directions.

As shown in FIG. 6, the split cavity mold 34 may generally comprise a first half cavity mold plate 64 and a second half cavity mold plate 66 which, when joined together in juxtaposed fashion, form the split cavity mold 34 with a mold cavity 68 extending from an inlet end 70 of the mold 34 to a distal end 72 of the mold 34. Although a split cavity mold 34 is illustrated in FIG. 6, it should be understood that other types of mold structures may be used as well. Split cavity mold structures are disclosed in detail U.S. Pat. No. 7,047,608 to Sageser, et al. and pending U.S. patent application Ser. No. 10/887,645 entitled “Compressed, Gas Stabilized Tampon Having Multiple Folds” filed on Jul. 9, 2004.

The mold cavity 68 of the split cavity mold 34 may have a cross-sectional shape similar to, and, in certain embodiments, substantially identical to the cross-sectional shape of the compression machine cavity 60 when in the compressed configuration. Furthermore, the mold cavity 68 may have a length L4 extending from the inlet end 70 to a distal end 72 of the mold cavity, a width W4 extending substantially perpendicularly to the mold cavity length L4, and a thickness T4 extending substantially perpendicularly to both the length L4 and width W4 of the mold cavity 34. In accordance with certain embodiments, the thickness T4 of the mold cavity 68 may be no more than about 10% different than the thickness T3 of the compressed pledget 33, or no more than about 5% different than the thickness T3 of the compressed pledget 33, or no more than about 2% different than the thickness T3 of the compressed pledget 33. Also, in certain embodiments, the width W4 of the mold cavity 68 may be no more than 10% different than the width W3 of the compressed pledget 33, or no more than about 5% different than the width W3 of the compressed pledget 33, or no more than about 2% different than the width W3 of the compressed pledget 33. Thus, in certain embodiments, the width W4 and thickness T4 of the mold cavity 68 may be close to the width W3 and thickness T3 of the compression machine cavity 60 when in the compressed configuration. Therefore, the thickness T4 of the mold cavity 68 may be no more than about 10% different than the thickness T3 of the compression machine cavity 60, or no more than about 5% different than the thickness T3 of the compression machine cavity 60, or no more than about 2% different than the thickness T3 of the compression machine cavity.

In certain embodiments, the width W4 of the mold cavity 68 may be no more than about 10% different than the width W3 of the compression machine cavity 60, or no more than about 5% different than the thickness T3 of the compression machine cavity, or no more than about 2% different than the width W3 of the compression machine cavity. Because cross-sectional shape and dimensions of the compression machine cavity 60 and the compressed configuration are very similar to, and, in certain embodiments, substantially identical to the cross-sectional shape and dimensions of the mold cavity 68, the compressed pledget 33 may not expand or otherwise change shape significantly when inserted directly from the compression machine cavity 60 into the mold cavity 68 and, as a result, the shallow creases of the compressed pledget 33 are maintained.

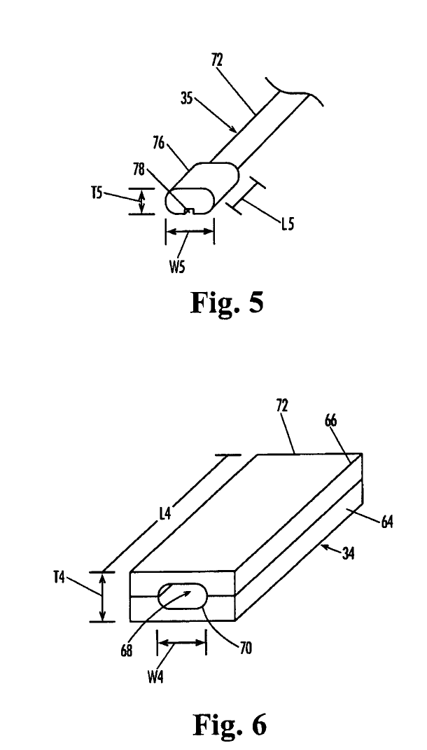

As shown in FIG. 5, the compression member 35 may comprise an actuating rod 74 and a head 76 connected to the actuating rod 74 for contacting the compressed pledget 33 to push the compressed pledget 33 from the compression machine cavity 60 into the mold cavity 68. The cross-sectional shape of the compression member head 76 may be similar to and, in certain embodiments, substantially identical to the cross-sectional shape of the compression machine cavity 60 in the compressed configuration and the cross-sectional configuration of the mold cavity 68. In certain embodiments, the compression member head 76 may have a width W5 and a thickness T5 extending perpendicularly to the head width. In certain embodiments, the head width W5 may be no more than 10% less than the mold cavity width W4, or no more than about 5% less than the mold cavity width W4, or no more than about 2% less than the mold cavity width W4. In certain embodiments, the head thickness T5 may be no more than about 10% less than the mold cavity thickness T4, or no more than about 5% less than the mold cavity thickness T4, or no more than about 2% less than the mold cavity thickness T4.

The compression member head 76 may have a slot 78 therein for receiving the withdrawal cord 22 of the compressed pledget 33 so that the withdrawal cord is not cut by the compression member head 76 when the compression member head 76 transfers the compressed pledget 33 into the mold cavity 68.

Accordingly, the compressed pledget 33, the compression machine cavity 60 in the compressed configuration, the mold cavity 68, and the compression member head 76, each may have cross-sectional shapes and dimensions which are very similar and, in certain embodiments, even substantially identical. These close tolerances may help avoid trapping of fibers from the compressed pledget 33 as the compression member head 76 transfers the compressed pledget into the mold cavity 60. Trapped fibers may create binding and shearing forces that may damage the tampon forming apparatus 32 or the compressed pledget 33, or both.

According to certain embodiments, the compressed pledget 33 may be heated in the mold cavity 68 to impart a self-sustaining shape to the compressed pledget 33 and resulting tampon. Methods of setting or stabilizing the tampon shape include heating the compressed pledget 33 with steam as disclosed in U.S. patent application Ser. No. 10/887,645 or thermal temperature gradient conduction or microwaving, as disclosed in U.S. Pat. No. 7,047,608.

A variety of materials may be used to make the components of the tampon forming apparatus 30. Suitable materials may be relatively rigid and include, but are not limited to stainless steel, and in the case of microwave heat stabilization, microwave safe materials.

C. Method of Making Tampons

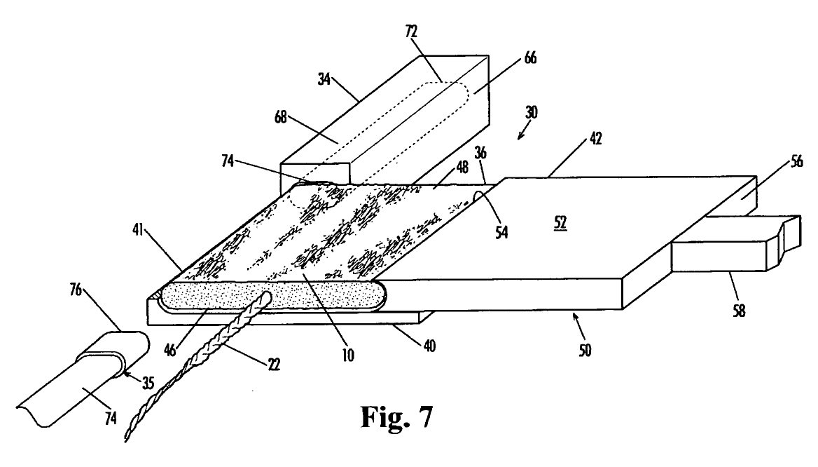

A tampon may be made in accordance with an embodiment of this invention by first inserting the uncompressed pledget 10 in the open compression machine cavity 60 as shown in FIG. 7. As described hereinbefore, the thickness T1 of the uncompressed pledget 10 may be very close to the thickness T2 of the compression machine cavity 60 and the width W1 of the uncompressed pledget 10 may be close to or less than the width W2 of the compression machine cavity 60. The length L1 of the uncompressed pledget 10, however, may be less than the length L2 of the compression machine cavity 60.

According to certain embodiments, the thickness of the uncompressed pledget 10 can vary as can the particular dimensions of the compression machine cavity 60, mold cavity 68, and compression member head 76, but, according to certain embodiments, uncompressed pledget 10 thickness may generally range from about 5 mm to about 15 mm, or from about 5 mm to about 12 mm, or from about 5 mm to about 9.8 mm.

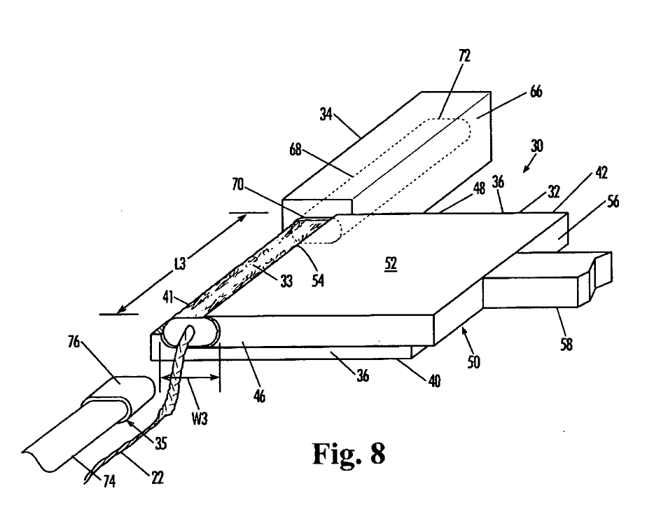

The uncompressed pledget 10 may then compressed in the compression machine cavity 60 by actuating the die 50 of a tampon compression machine 32 within the anvil channel 44 toward the end wall 41 of the anvil 36 until the compressed configuration illustrated in FIGS. 4 and 8 is reached. The amount of force required to compress the pledget 10 may vary but suitable forces typically are from about 40 psi to about 300 psi. A variety of techniques for actuating the compression die 50 are well known and may include, but are not limited to a modified tampon compression machine available from Tory Engineering Company, of Osaka, Japan. According to certain embodiments, the compressed pledget width W3 is predetermined and the compression machine 32 compresses the uncompressed pledget 10 only to the compressed pledget width W3. In accordance with certain embodiments, methods for stopping the compression applied by the die 50 may include, but are not limited to a stop or détente structure for stopping forward movement of the die 50 when the predetermined compressed pledget width W3 is reached or suitable controls on the actuating mechanism for reciprocating the die 50.

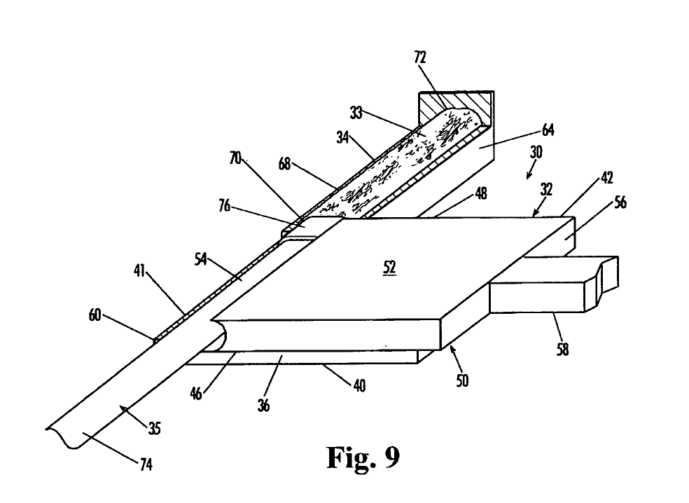

After compression in the tampon compression machine 32, the compressed pledget 33 may be ejected from the compression machine cavity 60 by actuating the compression member 35 so that the compression member head 76 enters the inlet end 46 of the compression machine cavity and extends through the compression machine cavity 60 forcing the compressed pledget 33 through the inlet end 70 of the mold cavity 68 until the compressed pledget 33 compacts against the distal end 72 of the mold cavity 68 and the compressed pledget 33 is completely within the mold cavity 68 as shown in FIG. 9. Once in the mold cavity 68, the compressed pledget 33 may be stabilized by conventional heat treatment and then ejected from the mold cavity by conventional means such as by pulling the withdrawal cord 22, manually or mechanically by grasping, hooking, picking, or clamping the tampon and withdrawing it from the mold, or vacuum withdrawal, or the like. Suitable methods of tampon removal from the mold are described in U.S. Pat. No. 7,047,608.

D. Tampons

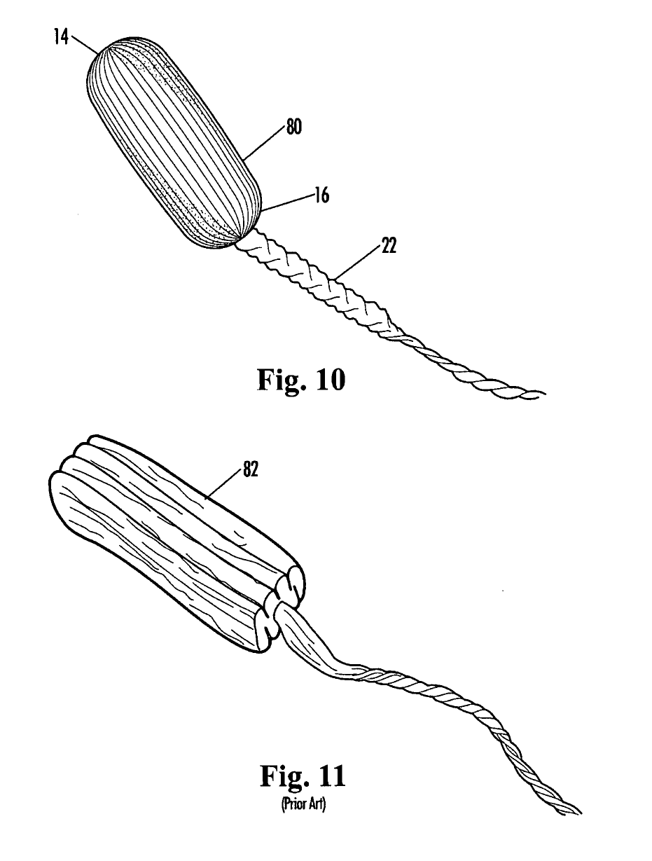

Tampons made by the foregoing method may have shallow creases created in the outer surface of the tampon. A tampon 80 made in accordance with an embodiment of this invention is illustrated in FIG. 10 and this can be compared to an illustration of a prior art tampon 82 illustrated in FIG. 11. The tampon 80 illustrated in FIG. 10 is made according to an embodiment of this invention and may have a plurality of creases extending along the length and across the width of the tampon from the surface of the tampon, a majority of such creases penetrating no deeper than about 20% of the width of the tampon. In certain embodiments, all the creases but a crease created by the withdrawal cord 22 penetrate no deeper than about 20% of the tampon width.

The tampon 80 as illustrated in FIG. 10 and the tampons made according to the embodiments of this invention are made by applying a compression to the uncompressed pledget 10 having a major compression component in a widthwise direction with respect to the tampon and no more than a minor component of compression in a lengthwise and/or thickness direction with respect to the tampon. This is explained in detail in the foregoing description of the tampon forming apparatus 30. Prior art processes which manufacture tampons by applying a major component of compression in the lengthwise direction with respect to the tampon and a minor component in a widthwise direction with respect to the tampon or which manufacture tampons with radial compression are distinct from the process of this invention. Other prior art processes which manufacture tampons by applying a major component of compression in the widthwise direction with respect to the tampon and a minor component in a lengthwise direction with respect to the tampon, but allow deep folds to occur within the tampon such that folds penetrate from more than 20% of the thickness of the tampon are also distinct from the method of this invention.

Table 1 below characterizes several commercially available prior art tampons and, as can be seen, those that have a major component of compression in the widthwise direction have fewer, deeper folds than do the embodiments of this invention:

TABLE 1
Commercially available tampons

| | Compression | Compression | |
|---|---|---|---|
| Tampon | Major | Minor | Surface |
| Kotex | Length | Width | Multiple folds penetrating |
| | | | 50% of width |
| Rostam | Length | Width | Multiple folds penetrating |
| | | | 35% of width |
| Tampax | Length | Width | 3 folds penetrating 50% of |
| | | | width |
| New Tampax | Width | Length | 3 to 5 folds penetrating |
| | | | 50% of width |
| First Quality | Width | Length | 5 folds penetrating 35% of |
| | | | width |
| Pearl | Width | Length | 3 to 5 folds penetrating |
| | | | 50% of width |
| Playtex Sport | Radial | Length | No folds visible from high |
| | | | density, but folds penetrate |
| | | | 50% of width |
| Playtex Beyond | Radial | Length | 4 channels penetrating 30% |
| | | | of width |
| Playtex Gentle | Radial | Length | No folds visible, but folds |
| Glide | | | penetrate 45% of width |
| o.b. | Radial | Length | 6–8 channels penetrating |
| | | | 30% of width |

In certain embodiments of this invention, the uncompressed pledget may be compressed with a compression having a major component in a widthwise direction within a range from about 65% to about 90% compression with respect to the compressed or finished tampon and no more than a minor component in a lengthwise direction within a range from about 20% to about 40% with respect to the compressed or finished tampon. In certain embodiments, the uncompressed pledget may be compressed with a compression having a major component in a widthwise direction within a range from about 75% to about 85% compression with respect to the compressed or finished tampon and no more than a minor component in a lengthwise direction within a range from about 20% to about 30% with respect to the compressed or finished tampon.

In accordance with certain embodiments, a majority of the plurality of creases in the tampon 80 penetrate no deeper than about 20% of the tampon thickness, or no deeper than about 15% of the tampon width, or no deeper than about 10% of the tampon width. As used herein, “majority” means more than half. In certain embodiments, all the creases but a crease created by the withdrawal cord 22 penetrate no deeper than the above described portion of the tampon width. In accordance with certain embodiments of the invention, the plurality of creases in the tampon may be at least 8 in number, or range from eight to twenty in number. According to certain embodiments, the compressed pledget of the tampon may have a density in the range from about 0.32 g/cc to about 0.60 g/cc or from about 0.34 g/cc to about 0.40 g/cc.

According to a certain embodiment of the invention, a tampon may have an aspect ratio of width to thickness greater than about 1.4:1, a width of about 17 mm and an expanded width upon fluid absorption of at least about 25 mm, a difference between tampon width when compressed and the expanded width upon fluid absorption of at least about 8 mm, or a difference between the tampon width when compressed and the expanded width upon fluid absorption of at least about 40% as determined by the Expansion Under Pressure Test described hereinbelow, or a combination of the foregoing.

According to one embodiment, a tampon may be made by compressing in the tampon compressing machine 32 an uncompressed pledget 10 having a width of about 70 mm, a length of about 64 mm, and a thickness of about 8 mm. The compression machine cavity 60 may have a length of about 100 mm, a thickness of about 9.8 mm, and a width in the open configuration of about 80 mm. After compression, the compressed pledget 33 may have a length of about 64 mm, a thickness of about 9.8 mm, and a width of about 16.9 mm. The compression member head 76 may have a width of about 16.7 mm and a thickness of about 9.6 mm and a slot 78 for the withdrawal cord 22 having dimensions of about 0.45 mm deep and about 1.0 mm wide. Furthermore, the mold cavity 68 and the resulting tampon may have a width of about 16.9 mm and a thickness of about 9.8 mm and the compression member head 76 may further compress the compressed pledget 33 such that the resulting tampon may have a length of about 48 mm.

### Expansion Under Pressure Test:

This test is a modification of the standard syngyna test which is well known to those skilled in the art. This test may be used to determine the widthwise expansion under pressure of tampons made according to the present invention. Additionally, this test produces measurements of tampon width as a function of time from the start of the test. These measurements may be used to calculate a widthwise expansion rate by dividing the width at a given time interval minus the width at time zero by the total time elapsed in such time interval.

### Procedures:

1. Use the following equipment.
a) Ring stand
b) Clamp, chain; VWR #21573-275
c) Calibrated syngyna chamber
d) Clamp, swivel; 21572-603 VWR
e) Compressed air station with PSI gauge
f) 40 inches of 6409-13 tubing (size 13; Tygon)
g) Steel cylinder standards

### h) Condoms, Calatex

i) Steel cannula, peristaltic pump head and drive motor

### j) Beakers

k) Traceable timer

### l) Rule

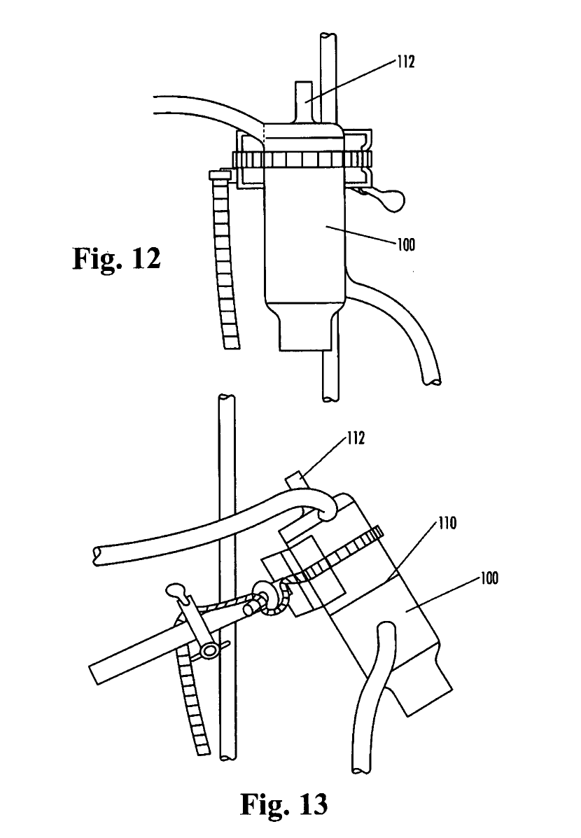

m) 06429-18 tubing for air pressurizing of chamber ⅜″ I.D.
n) Tubing clamp
o) Digital camera
p) Leveling protractor
q) KLC 9-25-00 not needed
2. Set up equipment as pictured in FIG. 12.
3. Setup tripod and camera in front of syngyna chamber 100. Place camera as close to the chamber as possible while still being able to see the entire chamber in the view finder.
4. Adjust angle of chamber to 30° from upright (60° on protractor) as shown in FIG. 13.
5. Adjust angle of camera to 30° so that it is parallel to the chamber. Looking through view-finder, the calibration line 110 should be even and solid.
6. Assemble pump head and motor; insert tubing; insert cannula into tubing.
7. Insert a condom into the syngyna chamber, cut off tip and secure top and bottom around ends of chamber with rubberbands. (Same procedure as in syngyna method). Place small rule inside chamber in front of condom, then secure bottom of condom around opening of chamber.
8. Turn on pump motor and dispense test fluid (Sheep's blood, definbrinated) for a set period of time into a tared beaker. Weigh beaker and determine flow rate. Target is 1 gram per minute.
9. Insert tampon into chamber, centering it using calibration line 110.
10. Close clamp on air tube and turn on air pressure. Adjust to 0.25 psi.
11. Insert cannula into top of chamber 112. Be sure it touches top of tampon.
12. Check angle of chamber again. Check set-up by looking through viewfinder of camera. Be sure everything is straight and level. Be sure timer is visible in frame.
13. Take a picture of dry tampon in chamber. This will be time=0.
14. Start pump and timer simultaneously.
15. Take a picture of tampon each minute until it leaks.
16. At leak point, release pressure in chamber and remove tampon.
17. Download images to computer.
18. Using ScionImage analysis software, open each image and measure at least one or two rules. That is, use the measurement line to draw a line over a certain number of millimeters on the rule in an image. Then, select “Analyze” on menu bar, then “set scale”. Type in number of millimeters measured in image. The software will then set a pixels per-millimeter scale.
19. Using the same measurement line tool, measure the tampon in the image. Measure the widest portion of the tampon as well as the width at the top and bottom of the tampon. For purposes of this instruction, the “top” of the tampon to the widest part above the calibration line on the chamber. The “bottom” is approximately 7 mm from the bottom most edge of the tampon.
20. Record measurements.
21. Verification measurements can be made on known standards such as cylinders.
22. Special notes: Periodically check angle of camera and be sure set-up is not disturbed. Check angle of chamber after insertion of each tampon. While it is not necessary to set scale for each image, it is recommended to do so frequently. It is recommended to check scale by measuring rule in image at least every two images.
This concludes the Expansion Under Pressure Test.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

All documents cited in the Detailed Description of the Invention are, in relevant part, incorporated herein by reference; the citation of any document is not to be construed as an admission that it is prior art with respect to the present invention. To the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A tampon comprising a compressed pledget of absorbent material, the tampon having a surface, a length extending from a withdrawal end to an insertion end distal from the withdrawal end, a width extending substantially perpendicularly with respect to the length, a thickness extending substantially perpendicularly with respect to the length and width, and a plurality of creases extending along the length of the tampon with a majority of the creases penetrating from the surface no deeper than about 20% of the width of the tampon, the compressed pledget having been compressed with a major component in a widthwise direction with respect to the tampon and no more than a minor component in lengthwise and thickness directions with respect to the tampon.

2. A tampon as in claim 1 wherein a majority of the plurality of creases penetrate no deeper than about 15% of the tampon width.

3. A tampon as in claim 1 wherein a majority of the plurality of creases penetrate no deeper than about 10% of the tampon width.

4. A tampon as in claim 1 wherein the plurality of creases are at least 8 in number.

5. A tampon as in claim 1 wherein the plurality of creases are from 8 in to 20 in number.

6. A tampon as in claim 1 wherein the difference between the tampon width when compressed and the expanded width upon fluid absorption is at least about 40%.

7. A tampon as in claim 1 wherein the major component of compression in the widthwise direction is within a range from about 65% to about 90% compression with respect to the compressed or finished tampon and the minor component of compression in the lengthwise direction is within a range from about 5% to about 40% compression with respect to the compressed or finished tampon.

8. A tampon as in claim 1 wherein the major component of compression in the widthwise direction is within a range from about 75% to about 85% compression with respect to the compressed or finished tampon and the minor component of compression in the lengthwise direction is within a range from about 5% to about 30% compression with respect to the compressed or finished tampon.

9. An apparatus for making a tampon comprising:
a compression machine for receiving and compressing an uncompressed pledget of absorbent material, the compression machine structured for compressing the uncompressed pledget with a compression having a major component in a widthwise direction with respect to the uncompressed pledget and no more than a minor component in lengthwise and thickness directions with respect to the uncompressed pledget so as to form a compressed pledget having a compressed pledget length extending from an insertion end to a withdrawal end, a compressed pledget width substantially perpendicular to the compressed pledget length, and a compressed pledget thickness extending substantially perpendicularly to both the compressed pledget length and the compressed pledget width;
a tampon mold having a mold cavity for receiving the compressed pledget from the compression machine, the mold cavity having a mold cavity length extending from an opening to a distal end, a mold cavity width extending substantially perpendicularly to the mold cavity length, and a mold cavity thickness extending perpendicularly to both the mold cavity length and mold cavity width, the mold cavity thickness being no more than about 10% different than the compressed pledget thickness; and
a compression member for pushing the compressed pledget into the tampon mold cavity.

10. An apparatus as in claim 9 wherein the mold cavity width is no more than about 10% different than the compressed pledget width.

11. An apparatus as in claim 9 wherein the compression member comprises a head for contacting the compressed pledget and pushing the compressed pledget from within the compression machine to within the tampon mold, the head having a head width and a head thickness extending perpendicularly to the head width, the head width being no more than about 10% less than the mold cavity width and the head thickness being no more than about 10% less than the mold cavity thickness.

12. An apparatus as in claim 9 wherein the compression member head has a slot for receiving a withdrawal cord of the compressed pledget.

13. An apparatus as in claim 9 wherein the compression machine comprises a compression machine cavity having a compression machine cavity length, a compression machine cavity width extending perpendicularly to the compression machine cavity length, and a compression machine cavity thickness extending perpendicularly to both the compression machine cavity length and the compression machine cavity thickness, the compression machine cavity thickness being no more than about 10% different than the mold cavity thickness.

14. A method for making a tampon comprising:
providing an uncompressed pledget of absorbent material, the uncompressed pledget having an uncompressed pledget length extending from an insertion end to a withdrawal end, an uncompressed pledget width substantially perpendicular to the uncompressed pledget length, and an uncompressed pledget thickness extending substantially perpendicularly to both the uncompressed pledget length and the uncompressed pledget width;
compressing the uncompressed pledget in a compression machine with a compression having a major component in a widthwise direction with respect to the uncompressed pledget and no more than a minor component in thickness and lengthwise directions with respect to the uncompressed pledget so as to form a compressed pledget having a compressed pledget length extending from the insertion end to the withdrawal end, a compressed pledget width substantially perpendicular to the compressed pledget length, and a compressed pledget thickness extending substantially perpendicularly to both the compressed-pledget length and the compressed pledget width, the compressed pledget thickness being no more than about 20% different than the uncompressed pledget thickness; and
feeding the compressed pledget with a compression member into a tampon mold having a mold cavity for receiving the compressed pledget from the compression machine, the mold cavity having a mold cavity length extending from an opening to a distal end, a mold cavity width extending substantially perpendicular to the mold cavity length, and a mold cavity thickness extending perpendicular to both the mold cavity length and mold cavity width, the mold cavity thickness being no more than about 10% different than the compressed pledget thickness.

15. The method as in claim 14 wherein the compressed pledget thickness is no more than about 10% different than the uncompressed pledget thickness.

16. The method as in claim 14 wherein the mold cavity width is no more than about 10% different than the compressed pledget width.

17. The method as in claim 14 wherein the compression member comprises a head and the step of feeding the compressed pledget to the mold cavity further comprises contacting the compressed pledget and pushing the compressed pledget with the compression member head into the tampon mold, the head having a head width and a head thickness extending perpendicularly to the head width, the head width being no more than about 10% less than the mold cavity width and the head thickness being no more than about 10% less than the mold cavity thickness.

18. The method as in claim 14 wherein the compression machine comprises a compression machine cavity having a compression machine cavity length, a compression machine cavity width extending perpendicularly to the compression machine cavity length, and a compression machine cavity thickness extending perpendicularly to both the compression machine cavity length and the compression machine cavity thickness, the compression machine cavity thickness being no more than about 10% different than the mold cavity thickness.

19. The method as in claim 14 wherein the compression machine compresses the uncompressed pledget within a range from about 65% to about 90% in the widthwise direction and about 40% or less in the lengthwise direction.

20. The method as in claim 14 wherein the compressed pledget width is predetermined and the compression machine compresses the uncompressed pledget only to the compressed pledget width.
